(19) United States
(12) Patent Application Publication
(10) Pub. No.: US 2010/0069292 A1
(43) Pub. Date: Mar. 18, 2010

(54) INSULIN WITH A BASAL RELEASE PROFILE

(75) Inventors: Roderike Pohl, Sherman, CT (US); Nandini Kashyap, Danbury, CT (US); Robert Hauser, Columbia, MD (US); Koray Ozhan, Milford, CT (US); Solomon S. Steiner, Mount Kisco, NY (US)

Correspondence Address: Pabst Patent Group LLP, 1545 PEACHTREE STREET NE, SUITE 320, ATLANTA, GA, 30309, US

(73) Assignee: Biodel, Inc.

(21) Appl. No.: 12/552,855

(22) Filed: Sep. 2, 2009

Related U.S. Application Data
(60) Provisional application No. 61/238,024, filed on Aug. 28, 2009.
(60) Provisional application No. 61/142,596, filed on Jan. 5, 2009.
(60) Provisional application No. 61/093,604, filed on Sep. 2, 2008.

(51) Int. Cl. A61K 38/28

(52) U.S. Cl. 514/3

(57) ABSTRACT

A clear basal insulin formulation composed of insulin (preferably human recombinant insulin), buffering agents, precipitating agents, and/or stabilizing agents for subcutaneous, intradermal or intramuscular administration. The formulation is designed to form a precipitate of insulin following injection, creating a slow releasing “basal insulin” over a period of 12 to 24 hours, which can be varied by compositional changes to tailor the release profile to the needs of the individual diabetic patient

### PRIORITY CLAIM

This application claims priority to U.S. Ser. No. 61/093,604 “Insulin with a Basal Release Profile” filed Sep. 2, 2008 by Roderike Pohl, Solomon S. Steiner and Nandini Kashyap, U.S. Ser. No. 61/142,596 “Insulin with a Basal Release Profile” filed Jan. 5, 2009 by Roderike Pohl, Solomon S. Steiner and Nandini Kashyap, and U.S. Ser. No. 61/238,024 “Insulin with a Basal Release Profile” filed Aug. 28, 2009, by Roderike Pohl, Nandini Kashyap, Robert Hauser, Koray Ozhan, and Solomon S. Steiner.

### FIELD OF THE INVENTION

The present invention generally relates to formulations containing insulin in a formulation providing extended release of insulin following administration.

### BACKGROUND OF THE INVENTION

Glucose is a simple sugar used by all the cells of the body to produce energy and support life. Humans need a minimum level of glucose in their blood at all times to stay alive. The primary manner in which the body produces blood glucose is through the digestion of food. When a person is not getting sufficient glucose from food digestion, glucose is produced from stores in the tissue and released by the liver. The body's glucose levels are primarily regulated by insulin. Insulin is a peptide hormone that is naturally secreted by the pancreas. Insulin helps glucose enter the body's cells to provide a vital source of energy.

When a healthy individual begins a meal, the pancreas releases a natural spike of insulin called the first-phase insulin release. In addition to providing sufficient insulin to process the glucose entering the blood from digestion of the meal, the first-phase insulin release acts as a signal to the liver to stop making glucose while a meal is being digested. Because the liver is not producing glucose and there is sufficient insulin to process the glucose from digestion, the blood glucose levels of healthy individuals remain relatively constant and their blood glucose levels do not become too high.

Diabetes is a disease characterized by abnormally high levels of blood glucose and inadequate levels of insulin. There are two major types of diabetes—Type 1 and Type 2. In Type 1 diabetes, the body produces no insulin. In the early stages of Type 2 diabetes, although the pancreas produces insulin, either the body does not produce the insulin at the right time or the body's cells ignore the insulin, a condition known as insulin resistance.

Even before any other symptoms are present, one of the first effects of Type 2 diabetes is the loss of the meal-induced first-phase insulin release. In the absence of the first-phase insulin release, the liver will not receive its signal to stop making glucose. As a result, the liver will continue to produce glucose at a time when the body begins to produce new glucose through the digestion of the meal. As a result, the blood glucose level of patients with diabetes goes too high after eating, a condition known as hyperglycemia. Hyperglycemia causes glucose to attach unnaturally to certain proteins in the blood, interfering with the proteins' ability to perform their normal function of maintaining the integrity of the small blood vessels. With hyperglycemia occurring after each meal, the tiny blood vessels eventually break down and leak. The long-term adverse effects of hyperglycemia include blindness, loss of kidney function, nerve damage and loss of sensation and poor circulation in the periphery, potentially requiring amputation of the extremities.

Between two and three hours after a meal, an untreated diabetic's blood glucose becomes so elevated that the pancreas receives a signal to secrete an inappropriately large amount of insulin. In a patient with early Type 2 diabetes, the pancreas can still respond and secrete a large amount of insulin. However, this occurs at the time when digestion is almost over and blood glucose levels should begin to fall. This inordinately large amount of insulin has two detrimental effects. First, it puts an undue extreme demand on an already compromised pancreas, which may lead to its more rapid deterioration and eventually render the pancreas unable to produce insulin. Second, too much insulin after digestion leads to fat storage and weight gain, which may further exacerbate the disease condition.

Because patients with Type 1 diabetes produce no insulin, the primary treatment for Type 1 diabetes is daily intensive insulin therapy. The treatment of Type 2 diabetes typically starts with management of diet and exercise. Although helpful in the short-run, treatment through diet and exercise alone is not an effective long-term solution for the vast majority of patients with Type 2 diabetes. When diet and exercise are no longer sufficient, treatment commences with various non-insulin oral medications. These oral medications act by increasing the amount of insulin produced by the pancreas, by increasing the sensitivity of insulin-sensitive cells, by reducing the glucose output of the liver or by some combination of these mechanisms. These treatments are limited in their ability to manage the disease effectively and generally have significant side effects, such as weight gain and hypertension. Because of the limitations of non-insulin treatments, many patients with Type 2 diabetes progress over time and eventually require insulin therapy to support their metabolism.

Insulin therapy has been used for more than 80 years to treat diabetes. Intensive insulin therapy for diabetes involves providing a basal insulin, ideally present at a uniform level in the blood over a 24 hour period and a bolus or meal time (prandial) insulin to cover the added carbohydrate load from digestion concomitant with each meal.

In 1936, Hans Christian Hagedorn and B. Norman Jensen discovered that the effects of injected insulin could be prolonged by the addition of protamine obtained from the “milt” or semen of river trout. The insulin was added to the protamine and the solution was brought to pH 7 for injection. In 1946, Nordisk Company was able to form crystals of protamine and insulin and marketed it in 1950 as NPH (“Neutral Protamine Hagedorn”) insulin. NPH insulin has the advantage that it can be mixed with an insulin that has a faster onset to compliment its longer lasting action.

In the 1950's and 1960's high concentrations of zinc (greater than 2% zinc bound to amorphous insulin) were used to stabilize precipitated insulin, creating a prolonged insulin effect. These formulations created the lente, semi-lente and ultra lente formulations of long acting insulin, intended for basal use (U.S. Pat. No. 3,102,077 to Christensen; U.S. Pat. No. 2,882,203 to Petersen). However, due to the unpredictability of the insulin release profile, these basal formulations have gradually been replaced by formulations providing a more “peakless” profile.

Until very recently, and in many places today, basal insulin is usually provided by the administration of two daily doses of NPH insulin, separated by 12 hours. A patient eating three meals a day and using NPH insulin as the basal insulin requires five injections per day, one with each of three meals and two NPH insulin injections, one in the morning and the other at bedtime. To reduce the number of injections the patient must take, the morning dose of NPH insulin has been combined with a short acting insulin, (recombinant human insulin) or a rapid acting insulin analog, such as lispro. A typical combination is a 70% NPH to 30% rapid acting insulin analog mixture. As a result, the patient can reduce the number of injections from five per day to four per day. See, e.g., Garber, Drugs, 66 (1):31-49 (2006).

More recently insulin glargine, (trade name LANTUS®) a “very long-acting” insulin analog has become available. It starts to lower blood glucose slowly after injection and keeps working for up to 24 hours. It differs from human insulin by having a glycine instead of asparagine at position 21 and two arginines added to the carboxy-terminus of the beta-chain. Insulin glargine is formulated at pH 4, where it is completely water soluble. After subcutaneous or intramuscular injection, the pH increases, causing the drug to precipitate, with just a small amount now soluble. This ensures that small amounts of LANTUS® are released into the body continuously, giving a nearly peakless profile. LANTUS® consists of insulin glargine dissolved in a clear aqueous fluid (100 IU, 3.6378 mg insulin glargine, 30 micrograms zinc, 2.7 mg m-cresol, 20 mg glycerol 85%, and water to 1 ml).

Rosenstock, et al. (Diabetes Care. 31 (1):20-5 (2008)), reported that patients who took insulin glargine had a much lower risk of low blood glucose (hypoglycemia) than the patients who took NPH insulin because of the predictable insulin release. Insulin spikes in the plasma can lead to hypoglycemia. During the day hypoglycemia can result in loss of mental acuity, confusion, increased heart rate, hunger, sweating and faintness. At very low glucose levels, hypoglycemia can result in loss of consciousness, coma and even death. While sleeping, these symptoms are not evident, so the patient is not aware of the need to ingest food to increase the glucose levels in the blood. Therefore, the predictability of insulin release overnight is critical. According to the American Diabetes Association (ADA), insulin-using diabetic patients have on average 1.2 serious hypoglycemic events per year, many requiring hospital emergency room visits by the patients. Therefore, a reliable slow releasing insulin formulation is extremely important for treatment of diabetes.

Though the long acting analog Lantus® has had remarkable success in the clinic, its safety has been questioned, due to the changes in the amino acid sequences in this insulin analog.

Therefore, it is the object of the present invention to provide a reliable, basal insulin formulation composed of recombinant regular human insulin as an alternative to basal analog formulations.

It is another object of the present invention to provide a basal insulin with “adjustable” release properties that can be formulated to provide a range of release times, and optionally, to be modified to provide a prandial/basal release profile.

### SUMMARY OF THE INVENTION

The basal insulin formulation is a clear solution for subcutaneous or intramuscular injection, containing human recombinant, bovine or porcine insulin, or insulin analogs, a zinc compound and a pH buffering agent. The clear solution, once injected, precipitates into a sustained releasing basal insulin or prandial/basal profile. A prandial-basal formulation is described that may avoid the need to mix prandial and basal formulations.

In one embodiment, the formulation is provided as a clear solution for subcutaneous injection at a pH below the isoelectric point of the insulin. As the bodily fluids at neutral pH (7-7.4) mix with the insulin solution post injection, the pH of insulin rises. The formulation contains buffering components that sustain the pH around the isoelectric point of approximately pH 5.5, enhancing the precipitation of insulin into particles post injection. These precipitated insulin particles persist in the subcutaneous tissue, resulting in a sustained release of insulin over a controlled period of time, for example, 24 hours, as depicted in FIG. 1. In the preferred embodiment, a buffering agent such as sodium acetate and a precipitating enhancing agent such as zinc chloride are used to promote precipitation post injection.

In another embodiment, the clear insulin solution is formulated below the isoelectric point of insulin and has excipients added to change the solubility of insulin at physiological pH. Post injection, the rise in pH around insulin results in a precipitate at physiological pH. These precipitated insulin particles have a basal release profile. In the preferred embodiment, a solubility modifier such as arginine or histidine is combined with a precipitating enhancing agent, such as zinc chloride.

In a third embodiment, the insulin is formulated as a clear solution below the isoelectric point of insulin and has buffer added to sustain the insulin at the isoelectric point to induce precipitation, solubility modifying agents and precipitation enhancing agents to reduce solubility of the insulin at physiological pH. In this preferred embodiment, a buffer such as sodium acetate, a solubility modifiying agent such as arginine and/or histidine and a precipitation enhancing agent such as zinc chloride are used to create a suspension with a basal release profile.

In a fourth embodiment, the insulin formulation is prepared as a clear solution above the isoelectric point, at or above a pH of 7.7. Post injection, the reduction in pH results in precipitation of the insulin, creating a slow release basal profile. In this embodiment, a buffer such as trisodium citrate or sodium phosphate, and/or a solubility modifying agent such as arginine and/or histidine, and a precipitation enhancing agent such as zinc chloride, are used to create insulin particles post injection with a basal release profile.

In a fifth embodiment, the insulin formulation is prepared as a clear solution and post injection slowly precipitates, creating a prandial release of insulin followed by a basal release profile.

The release profiles can be varied by adjusting the pH, the amount and ratio of excipients, thereby providing a range of formulations to meet individual patient's needs, which is not possible with an insulin analog.

### BRIEF DESCRIPTION OF THE DRAWINGS

FIG. 1 is a diagram showing a possible mechanism of how the precipitation forms and slowly dissolves following administration using a buffer to prolong time through the isoelectric point of insulin.

FIGS. 2A and 2B are titration curves demonstrating the buffering effect of sodium acetate on insulin following dilution with an extracellular fluid buffer.

FIG. 3A shows the effectiveness of different amino acids (histidine, arginine, lysine) on the solubility of insulin. FIG. 3B is a graph of the solubility of insulin (mg/ml) as a function of pH (5.5, 6.5, 7 and 7.5).

FIGS. 4A, 4B and 4C compare the effect of concentration (0.5, 1, 2, and 2.5 mg/ml) of arginine (FIG. 4A), histidine (FIG. 4B), and lysine (FIG. 4C) on the solubility of insulin (in mg/ml) at pH 5.5, 6.5, 7 and 7.5.

FIG. 5 is a graph showing the decreased solubility of insulin (percent insulin in solution) following transition from a pH 7.7 to 7.5.

FIG. 6 is a graph of a mean plasma concentration (μIU insulin/ml) versus time (minutes) curve of a basal formulation containing Zinc chloride (3 mg/ml) with sodium acetate buffer (6 mg/ml) (dark squares) compared to insulin glargine (Lantus®) (dark circles) following subcutaneous administration to diabetic miniature swine.

FIG. 7 is a graph of a concentration (plasma insulin in μU/ml) versus time (minutes) curve of a basal formulation containing Zinc chloride (2.5 mg/mL) with (dark triangles) or without (dark squares) the addition of arginine (0.5 mg/ml).

FIG. 8 is a graph of a concentration (plasma insulin in μU/mL) versus time (minutes) curve of a basal formulation containing arginine (0.5 mg/mL) and zinc chloride (2.5 mg/mL, open squares) compared to insulin glargine (dark diamonds) following subcutaneous injection into diabetic miniature swine.

FIG. 9 is a graph of a concentration (plasma insulin in μU/mL) versus time (minutes) curve of a basal formulation containing arginine (0.5 mg/mL), acetate buffer (0.578 mg/mL) and zinc chloride (2.5 mg/mL) and m-cresol (0.5 mg/mL) (-X-) compared to insulin glargine (dark diamond) following subcutaneous injection to diabetic miniature swine.

FIG. 10 is a graph of a concentration (plasma insulin in μU/mL) versus time (minutes) curve of a prandial basal formulation containing histidine (2.5 mg/mL) and zinc acetate (2 mg/mL) following subcutaneous administration to miniature swine.

FIG. 11 is a graph of mean glucose infusion rate (mg/kg/min) verses time (min.) from a human clinical trial in patients with type 1 diabetes treated with insulin glargine (-I-) or insulin formulated with 3 mg/mL ZnCl₂ and 6 mg/mL NaAcetate (open circles).

### DETAILED DESCRIPTION OF THE INVENTION

### I. Definitions

As used herein, “a less soluble insulin” refers to an insulin or insulin analog that is less soluble than human recombinant insulin in extracellular fluid, such as Earle's balanced salt solution E2888 (Sigma Aldrich) at physiological pH (6.2-7.4) and body temperature (e.g. 37° C.).

As used herein, “insulin” refers to human or non-human, recombinant, purified or synthetic insulin or insulin analogues, unless otherwise specified.

As used herein, “human insulin” is the human peptide hormone secreted by the pancreas, whether isolated from a natural source or made by genetically altered microorganisms.

As used herein, “non-human insulin” is insulin but from a non-human animal source such as a pig or cow. Bovine and porcine insulins differ in several amino acids from human insulin, but are bioactive in humans.

As used herein, an “insulin analogue” is a modified insulin, different from the insulin secreted by the pancreas, but still available to the body for performing the same or similar action as natural insulin. Through genetic engineering of the underlying DNA, the amino acid sequence of insulin can be changed to alter its absorption, distribution, metabolism, and excretion (ADME) characteristics. Examples include insulin lispro, insulin glargine, insulin aspart, insulin glulisine, insulin detemir. The insulin can also be modified chemically, for example, by acetylation.

As used herein, “human insulin analogues” are altered human insulins which are able to perform a similar action as human insulin.

As used herein, a “precipitating agent” refers to a chemical that enhances the formation of an insulinprecipitate, “seeds” an insulin precipitate, modifies the solubility of insulin at physiological pH, or stabilizes the pH of the insulin at the isoelectric point to induce or maintain precipitation. As used herin, a “buffer” is a chemical agent able to absorb a certain quantity of acid or base without undergoing a strong variation in pH.

As used herein, an “insulin stabilizing agent” is an agent that physically and chemically stabilizes the insulin by preventing the formation of breakdown products reducing the potency of the insulin. Examples include zinc at low concentrations (50 μg/mL or lower concentrations), while zinc at high concentrations is used as a precipitating agent.

As used herin, a “precipitate enhancing agent” refers to agents that enhance the stability of precipated insulin particles. Zinc is both an insulin stabilizing agent and a precipitate stabilizing agent.

As used herein,“a prandial insulin” refers to an insulin or insulin formulation that provides a short term rapid release insulin and delivers an effective amount of insulin to a patient to manage the patient's blood glucose fluctuations following a meal. Typical prandial insulins include rapid-acting insulin analogs, which have a pharmacokinetic profile that closely resembles endogenous insulin.

As used herein, “a basal insulin” refers to an insulin or insulin formulation that provides levels of insulin over a period of time after administration of about 12 to 24 hours effective amount of insulin to manage the patient's normal daily blood glucose fluctuations in the absence of a meal.

As used herein, “a basal release profile” refers to the amount and rate of release of insulin from the formulation into a patient's systemic circulation. In a graph of the patient's mean plasma insulin levels over time, a basal release profile generally has a minimal peak (often referred to as “a peakless profile”) and slowly and continuously releases insulin for a prolonged period of time, such as twelve to twenty-four hours following administration. One example of a formulation with a basal release profile is LANTUS®.

As used herein, a “suspending agent” refers to a substance added to retard the sedimentation of suspended particles in liquids.

As used herein, an “excipient” refers to an inactive substance used as a carrier, to control release rate, adjust isotonicity or aid the process by which a product is manufactured. In such cases, the active substance is dissolved or mixed with an excipient.

As used herein, a “pharmaceutically acceptable carrier” refers to a non-toxic, inert solid, semi-solid or liquid that is not pharmaceutically active, which is mixed with the pharmaceutically active agent. Remington's Pharmaceutical Sciences Ed. by Gennaro, Mack Publishing, Easton, Pa., 1995 discloses various carriers used in formulating pharmaceutical compositions and known techniques for the preparation thereof.

### II. Composition

The compositions contain insulin and excipients for injection. In the preferred embodiment, the formulation is suitable for subcutaneous administration and is slowly released into the systemic circulation.

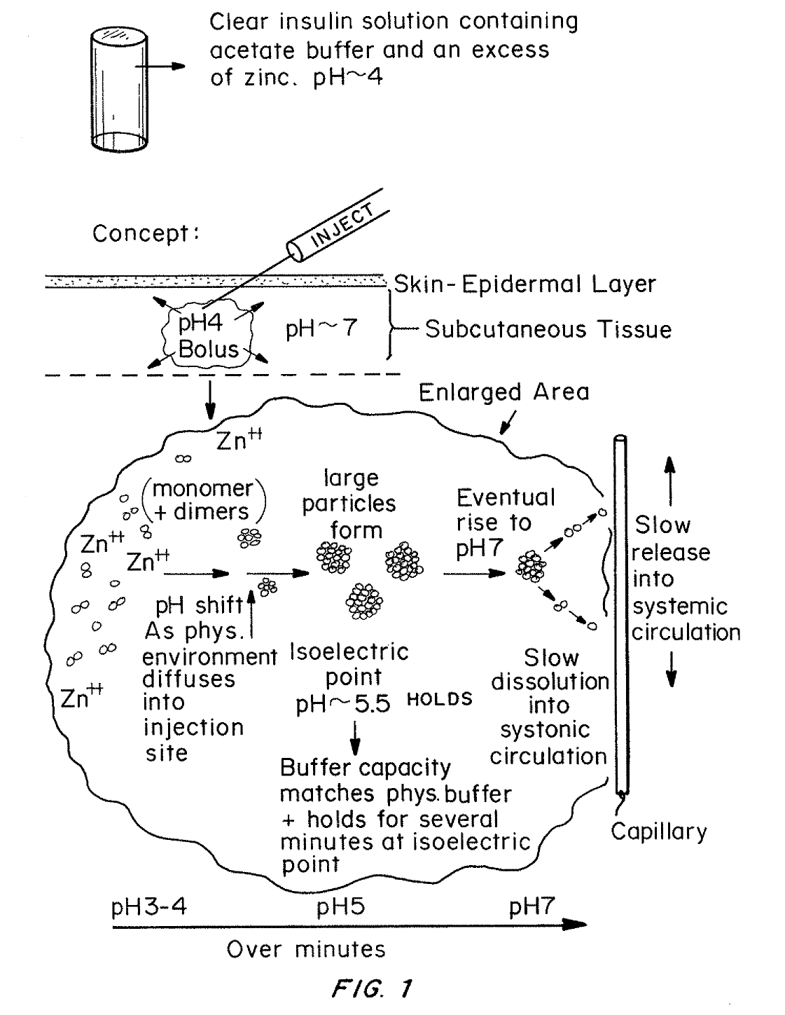

FIG. 1 is a schematic of a presumed mechanism of action. As shown in the top of FIG. 1, the insulin is administered as a clear solution of insulin, preferably 50 to 500 Units, in combination with a buffer such as a citrate or acetate (approximately pH 4), with an excess of zinc ions to maintain the insulin as a stable hexamer and enhance precipation. This is injected into the subcutaneous tissue or muscle. The tissue has a pH of about pH 7-7.2. As the pH of the injected insulin rises due to diffusion of the surrounding higher pH fluids, the insulin passes through its isoelectric point of about 5.5, creating a microprecipitate at the site of the injection. The buffer slows the progression to a pH of 7. The precipitated insulin then dissolves at a slow rate, and is absorbed through the capillaries, creating a basal systemic insulin profile.

A. Insulin

The insulin can be recombinant or purified from a natural source. The insulin can be human or non-human, such as porcine or bovine. In the preferred embodiment, the insulin is human recombinant insulin. The insulin may also be an insulin analogue which may be based on the amino acid sequence of human insulin but having one or more amino acids differences, or a chemically modified insulin or insulin analog.

Regular human insulin is commercially available as a pure white crystalline powder. It is made synthetically in large scale production, utilizing yeast or E. coli. The insulin precursor is grown in a fed-batch fermentor, which is released from the cells by lysis of their inclusion bodies. After refolding, the precursor is enzymatically cleaved to form a second insulin precursor. The second precursor is then purified chromatographically and enzymatically. This is then crystallized in the presence of zinc and washed with ethanol to produce a pure 52 amino acid final product.

B. Insulin Stabilizing Agents

Stabilizing agents are included in the formulation specifically to stabilize insulin as a hexamer in solution or reduce formation of B21 desamido which forms at pH 4 or other degradation products which form at neutral pH or above. An example is zinc at a concentration of 50 μg/mL or lower.

C. Precipitating Agents

Precipitating agents are added to enhance the formation of the insulin precipitate by either hastening the precipitate formation, and/or stabilizing the precipitate by reducing its solubility. These may be buffering agents, solubility modifying agents, precipitation seeding agents, or precipitation enhancing agents.

As the pH is increased from pH 4, towards physiological pH (7-7.5, typically 72-7.4), insulin transitions through its isoelectric point (pI) of about 5.5. The amount or form may be increased or form of the precipitate may be altered by increasing the residence time of the insulin at approximately its pI. This may be achieved by adding a buffering agent to the insulin formulation that is specifically selected for sufficient buffering capacity in the range of insulin's pI. Buffering agents include acetate, citrate, phosphate, carbonate, and barbital (FIG. 1). Preferred buffering agents are GRAS ingredients.

In one embodiment containing only a pH buffer, sodium acetate is used at a concentration ranging from 0.2 to 20 mg/mL, preferably from 1 to 10 mg/mL, most preferably 6 mg/mL. In another embodiment containing only a pH buffer where the insulin is present at a pH of about 8 as a clear solution and which forms a precipitate as the pH is dropped from 8 to 7 towards physiological pH, agents such as sodium phosphate or sodium citrate may be added to help form or stabilize the precipitate.

In a second embodiment, a charged molecule modifies the solubility of insulin at physiological pH. Examples of charged molecules (or solubility modifying agents) include amino acids such as arginine, histidine, lysine. A representative concentration of histidine ranges from 0.005 to 10 mg/mL, preferably from 0.5 to 2 mg/ml. A representative concentration of Arginine ranges from 0.005 to 10 mg/mL, preferably ranges from 0.25 to 2 mg/mL.

Precipitation “seeding” agents may be a solid nanoparticle or a molecule that precipitate at or near the pI of the insulin, thereby acting as a nucleation site for the insulin. Examples of nanoparticles include Au₁₂ (present in the formulation in a concentration range from 24 to 2400 ng/ml, preferably 240 ng/ml) and C₆₀ (present in the formulation in a concentration range from 75 to 7500 ng/ml, preferably 750 ng/mL). An example of a molecule that precipitates near the pI of insulin is cysteine with a pI of 5.0. An appropriate concentration of cysteine in the formulation ranges from 1.2 to 120 nM, and preferably is 12 nM.

Other precipitation enhancing agents are added to form or stabilize an insulin precipitate. Precipitation agents include various forms of zinc, calcium, magnesium, manganese, iron, copper, and other divalent ions used at non-toxic levels (range 0.1-10 mg/ml, preferably 2.5 mg).

These precipitation agents may be used individually or combined to modify the pharmacokinetics of insulin precipitation and solubilization following injection. Typically these precipitation agents are added so that all of the insulin is solubilized within 8 to 24 hours following administration. The formulation is designed to create the best conditions for precipitation post injection, leading to a stable micro-precipitate. The choice of agents is dependent on the intended duration of the formulation (e.g. typically the formulation is intended to release insulin for 8 to 24 hours following injection, preferably for 12 to 24 hours following injection) allowing the profile to be catered to individual patient's needs.

One of the benefits of the formulations is that the amount of precipitate and release rate following administration can be adjusted through the selection and amount of excipients such as the zinc salt and the pH buffer and/or amino acid. The insulin formulation can be provided in different compositions so that the physician can adjust the rate of release (See FIGS. 6-10). These will have different release rates by a few hours, and can be labeled “short”, “medium” and “long”. A physician can try different formulations and test blood glucose levels to determine which is best for that patient.

D. Other Excipients and Carriers

The formulations are administered by injection, preferably subcutaneous injection. The insulin is typically combined with pharmaceutically acceptable carriers such as sterile water or saline. Remington's Pharmaceutical Sciences Ed. by Gennaro, Mack Publishing, Easton, Pa., 1995 discloses various carriers used in formulating pharmaceutical compositions and known techniques for the preparation thereof.

In the preferred embodiments no excipients other than pH buffers, charged molecules and/or precipitating enhancers or stabilizers are added, although salts to make a solution isotonic, acid or base to adjust pH, colorants, and/or preservatives may be added.

In one embodiment, the combined insulin composition has a pH of about 3.5 to about 5.0, below the isoelectric point of the insulin or sufficiently above it to form a clear solution. Suitable pH modifying agents include, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium hydroxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, m-cresol, thimerosal, polysorbate 20 and combinations thereof.

Typically the insulin is dissolved or dispersed in a diluent to provide the insulin in a liquid form. Suitable diluents include, but are not limited to, water, buffered aqueous solutions, dilute acids, vegetable or inert oils for injection organic hydrophilic diluents, such as monovalent alcohols, and low molecular weight glycols and polyols (e.g. propylene glycol, polypropylene glycol, glycerol, and butylene glycol).

Typically the diluent also serves as a carrier for the insulin formulation.

The diluent typically contains one or more excipients. Examples of excipients in a typical diluent for an injectable formulation include glycols, salts, preservatives, and optionally a buffering agent. In the preferred embodiment, the diluent contains saline.

### III. Methods of Making the Formulations

In the preferred embodiment, the insulin formulation is made by combining all constituents into the diluent, and adjusting to a final pH to make a clear solution (pH approximately 4 or 8). The solution is sterile filtered and filled in a vial suitable for multiple injection dosing.

Alternatively, the insulin is provided in a kit containing one vial of insulin in lyophilized form and another vial to resuspend the insulin. The excipients may be present in one or both vials, as appropriate to adjust pH, and stabilize and buffer the formulation.

### IV. Methods of Using the Formulations

The formulations may be administered subcutaneously, intradermally or intramuscularly by injection. The formulation is designed to release basal amount of insulin following administration. Doses are administered once or twice a day, titered to each patient's individual requirements, based on glucose measurements and the patient's history. The typical dose of basal insulin is in the range of 0.3 U/kg/day, though severe diabetics can be dosed as much as 60 Units.

The present invention will be further understood by reference to the following non-limiting examples.

### EXAMPLES

### Example 1

### Demonstration of the Effectiveness of the Addition of Sodium Acetate in Holding an Insulin Solution in the Isoelectric Range During Dilution in Extracellular Fluid

Materials and Methods

In this experiment, the buffering effect of sodium acetate in a basal insulin formulation was demonstrated by monitoring the pH of the formulation with an automatic titrator while diluting the solution with synthetic extracellular fluid buffer (ECF). The purpose was to mimic the environment (pH and dilution) of the basal injection in vitro to determine if it was likely the clear solution would precipitate in situ as it transitioned through the isoelectric point.

Two different formulations were prepared. Formulation A contained 100 IU insulin and 3 mg/ml Zinc chloride. Formulation B contained 100 IU 3 mg/ml Zinc chloride and 6 mg/ml sodium acetate. Initial volume was 2 ml for both formulations. Then the formulations were titrated with ECF buffer to observe their pH profile.

Results

Formulation A, containing 100 IU insulin, 3 mg/ml Zinc chloride, reached pH: 7.0 by 2 fold dilution with ECF buffer. Formulation B, containing 100 IU insulin, 3 mg/ml Zinc chloride and 6 mg/ml sodium acetate, reached the same pH by 7 fold dilution. The buffering capacity of sodium acetate was shown clearly with the experiment.

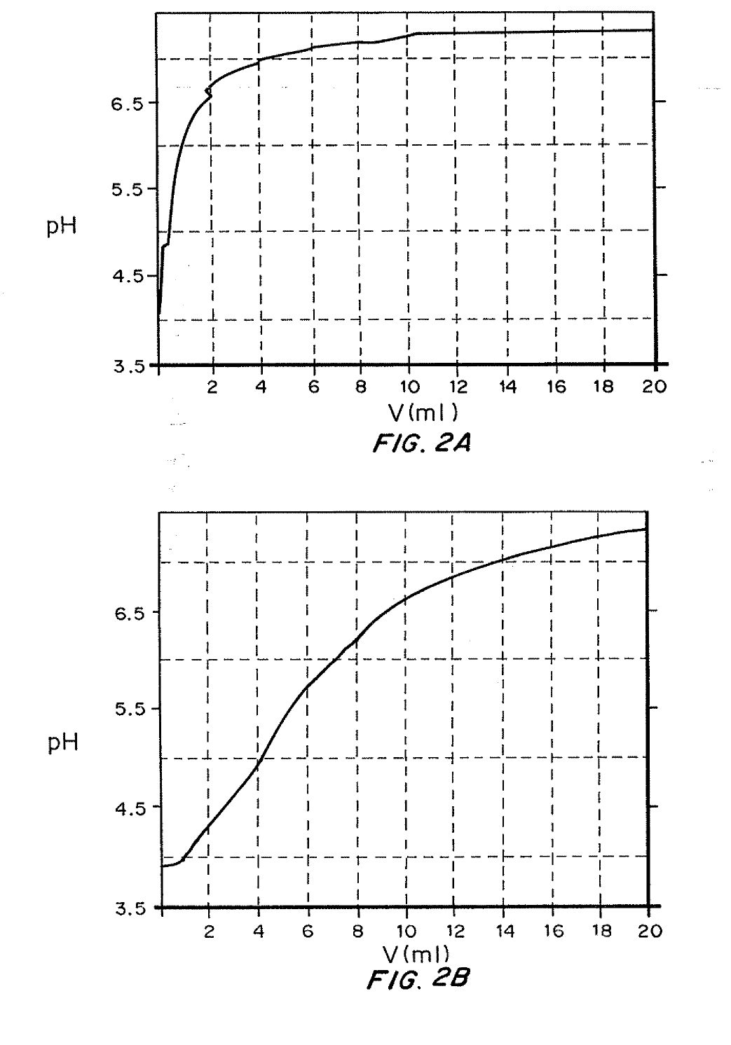

FIG. 2A and FIG. 2B show titration curves of the formulation A and B, respectively. The formulation was precipitated when exposed to extended periods at the isoelectric point and persisted at pH 7, while a rapid transition through the pH range resulted in a smaller precipitate that re-dissolved at pH 7.

In conclusion, the formulation containing sodium acetate has sufficient buffering capacity to create a persistent particulate insulin post injection.

### Example 2

### Comparison of Insulin Solubility at Various pH Using Different Amino Acids

Materials and Methods:

The purpose of this study was to identify the effect of various amino acids on the solubility of basal insulin formulations at a given pH and concentration.

The test formulation containing 2 mg/ml of Zinc Acetate, 0.5 mg/ml of Histidine, Arginine or Lysine and 100 U/ml insulin was prepared and adjusted to pH 4. Then the pH 4 formulations were adjusted to pH 5.5, 6.5, 7 or 7.5 and samples were centrifuged. For comparison, insulin alone was adjusted to pH 4, 4.5, 5, 4.5, 5, 5.5, 6, 6.5 or 7.

The quantity of insulin in the supernatant was determined by HPLC (High Performance Liquid Chromatography) analysis. The reverse phase chromatography was performed on a C-18 column, a mobile phase composition of 71 ml Water: 20 ml Acetonitrile: 9 ml Tetrahydrofuran and 0.1% TFA and a variable wavelength detector set at 210.0 nm. The HPLC acquisition parameters were: flow rate 1.0 ml/min, Sample Temp 5° C. and Column Temp 40° C. The insulin in the supernatant was measured by removing 0.5 mL sample and assaying by HPLC.

The relative to the initial amount of insulin in the solution was then determined. The soluble fraction is determined by centrifuging out the insoluble portion and assaying the remaining soluble insulin in the supernatant using HPLC. If the entire amount of insulin is measured in the supernatant, it is soluble, while if there is less insulin measured in the supernatant, then it must be in the precipitated material in the bottom of the test tube, hence “insoluble”

Results:

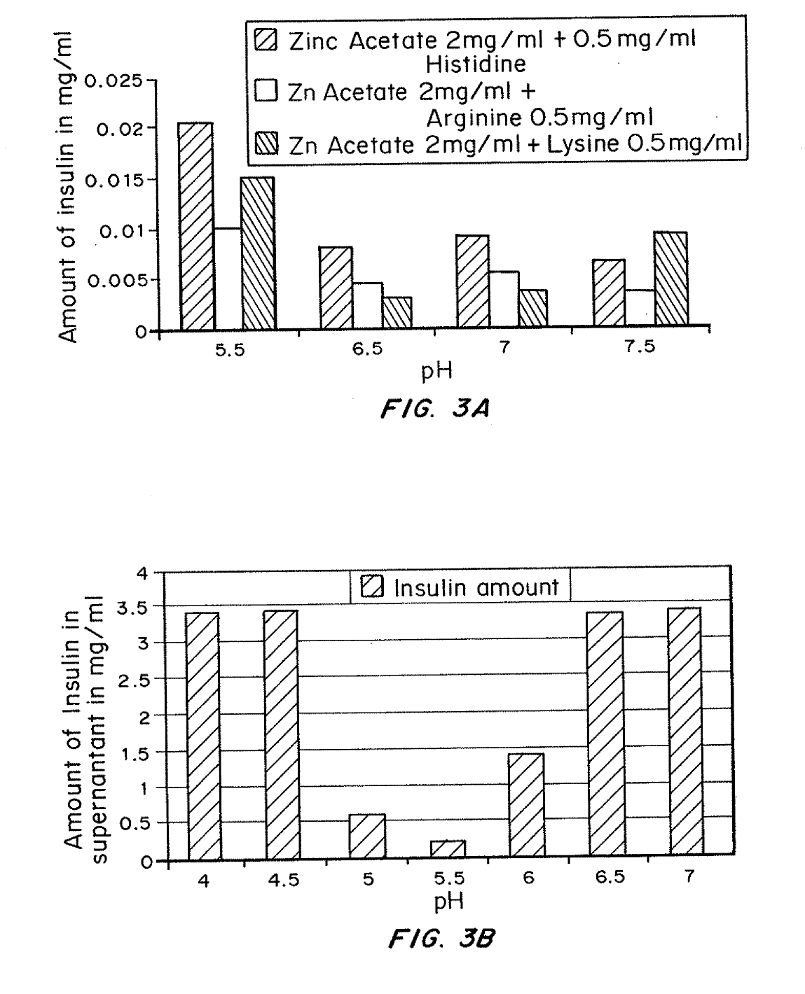

FIG. 3A shows the effect of various amino acids on the solubility of insulin at various pHs. The insulin amounts shown in FIG. 3A represent the soluble insulin fraction found in the supernatant at different pHs, with addition of 0.5 mg/ml of histidine, arginine or lysine.

Insulin is known to be soluble at higher pH (FIG. 3B). The results show that the addition of a small quantity (0.5 mg/ml) of histidine, arginine or lysine reduces the solubility of insulin even at higher pHs. At a given concentration and close to physiological pH, the formulation with Arginine shows the least soluble fraction of insulin, followed by the formulation with histidine, with the highest soluble fraction insulin in the formulation containing lysine. Overall, all of the formulations containing different amino acids had significantly reduced solubility of insulin at higher pH.

### Example 3

### Effect of pH on Solubility of Insulin in a Formulation Containing Insulin 100 IU/ml, Zinc Acetate 2 mg/ml, and Different Concentrations of Histidine 0.5 mg/ml, Arginine or Lysine

Materials and Methods:

The purpose of this study was to identify whether formulations containing zinc in combination with histidine, arginine or lysine would be less soluble as they precipitate and are exposed to increasing pH environments. This in vitro test was designed to illustrate the pH change of the environment following a subcutaneous injection in viva.

The test formulation containing 2 mg/ml of Zinc Acetate, various concentrations of Histidine or Arginine or Lysine and 100 U/ml insulin was prepared and adjusted to pH 4. Then the pH 4 formulation was adjusted to pH 5.5, 6.5, 7 and 7.5 and samples were centrifuged. The quantity of insulin in the supernatant was determined by HPLC (High Performance Liquid Chromatography) analysis. The reverse phase chromatography was performed on a C-18 column, a mobile phase composition of 71 ml Water: 20 ml Acetonitrile: 9 ml Tetrahydrofuran and 0.1% TFA and a variable wavelength detector set at 210.0 nm. The HPLC acquisition parameters were; flow rate 1.0 ml/min, Sample Temp 5° C. and Column Temp 40° C. were used. The insulin in the supernatant was measured by removing 0.5 mL sample and assaying by HPLC. The relative to initial amount of insulin in the solution was then determined. If the entire amount of insulin was measured in supernatent, it was all soluble, while if there was less insulin measured in the supernatant, then it must be in the precipitated material in the bottom of the test tube, hence “insoluble”.

Results:

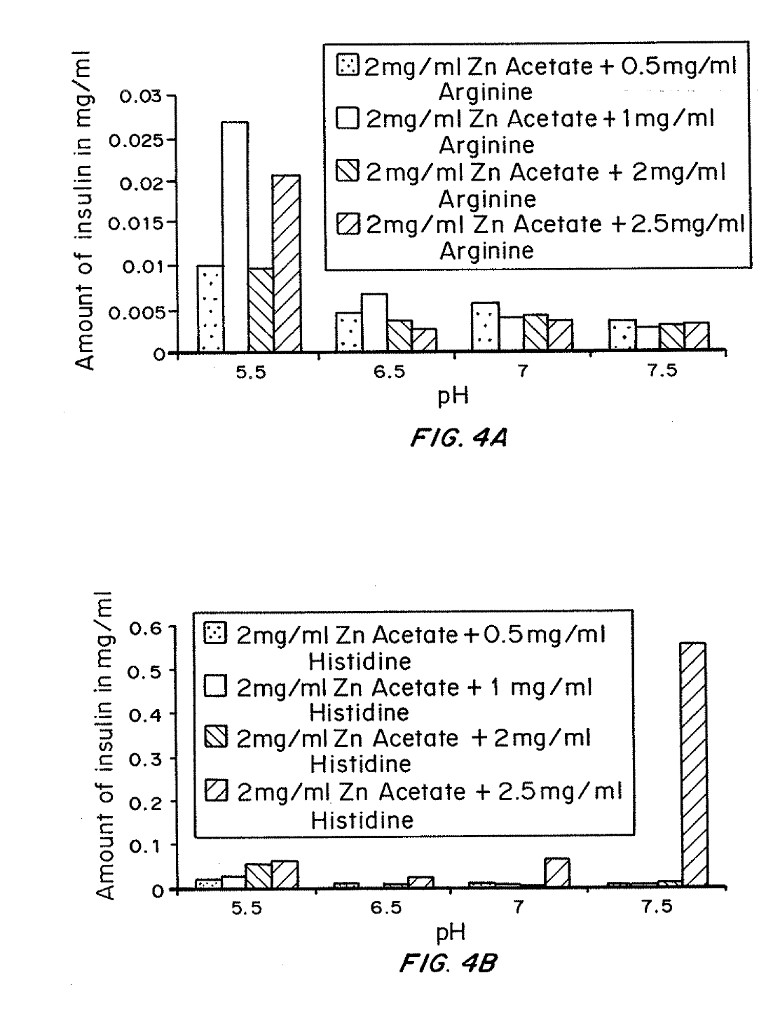

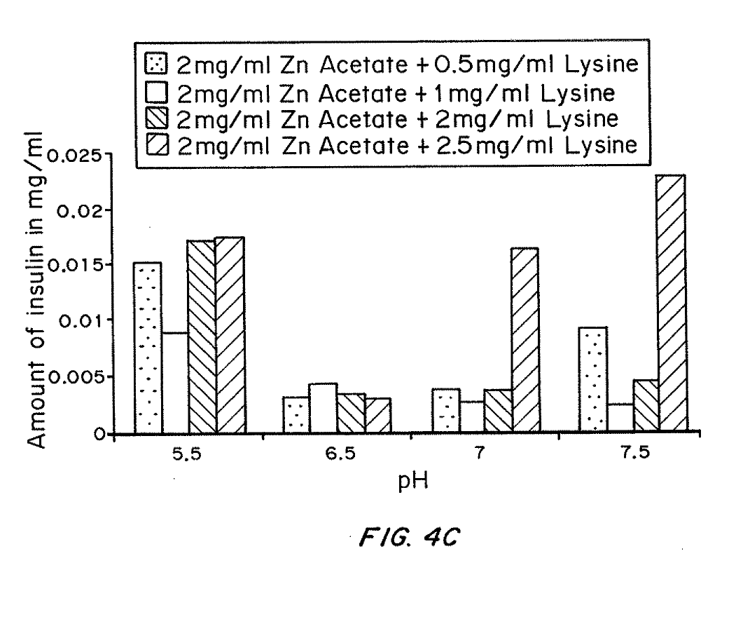

The insulin amounts shown in FIGS. 4A, 4B and 4C represent the soluble insulin fraction found in solutions of regular recombinant human insulin mixed at different pHs, compared to the addition of amino acids at different concentrations. The soluble fraction is determined by centrifuging the insoluble portion out and assaying the remaining soluble insulin in the supernatant.

The results show that the addition of histidine, arginine or lysine reduces the solubility of insulin even at higher pHs.

### Example 4

### Demonstration of Precipitation of a Clear Insulin Solution at pH 7.6 Upon Dilution in Extracellular Fluid Buffer, pH 7.2.

Materials and Methods:

An insulin formulation containing 100 U/mL insulin, 4 mg/ml trisodium citrate and 2.1 mg/ml ZnCl₂ was prepared at pH 7.65. The solution was subsequently diluted with extracellular fluid buffer (ECF) buffer, pH 7.2. The diluted solutions/suspensions were centrifuged to sediment the precipitated material. The supernatant was analyzed for insulin content by HPLC.

Results:

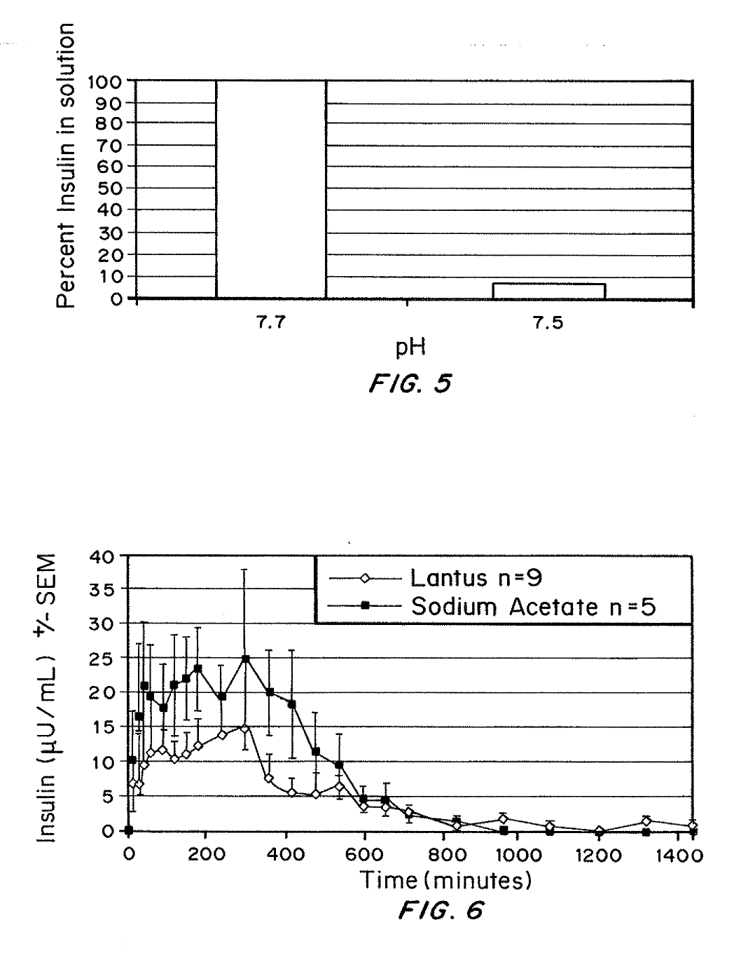

FIG. 5 shows the results of a 1:2 dilution with ECF buffer, which reduced the pH from 7.7 to 7.5. The insulin precipitated after the pH changed.

In conclusion, formulations containing zinc and buffer systems can be formulated to induce precipitation following a transition from high pH to the physiological range.

### Example 5

### Determination of Effect of Buffer on Basal Insulin Release in Diabetic Miniature Swine

Materials and Methods.

This example compares insulin activity of a formulation with insulin, zinc chloride and sodium acetate in diabetic swine. The purpose was to demonstrate that holding the pH at 5.5 in vivo by adding a buffer (sodium acetate) could extend the duration of insulin action.

Study Design:

0.45 U Insulin/kg was administered by subcutaneous injection to diabetic induced miniature swine. On dose administration, pigs were fed 500 g of their normal diet, and blood insulin and glucose were monitored for the following 24 hours.

Insulin Test Formulations:

1. Insulin 100 U/ml+Zinc chloride 3 mg/ml+Sodium acetate 6 mg/ml

2. Insulin glargine (Lantus)

Results:

FIG. 6 is a graph of mean insulin concentration versus time of a subcutaneous injection of the test basal formulation of insulin (squares) versus insulin glargine (diamonds). FIG. 6 demonstrates the effectiveness of the buffer in slowing down the release of insulin following injection, by keeping the insulin in the range of the isoelectric point until fully precipitated.

### Example 6

### Determination of Effect of Arginine on Basal Insulin Release in Diabetic Miniature Swine

Materials and Methods.

This example compares insulin activity of a formulation with insulin and zinc chloride, with and without arginine in diabetic swine. In another study, the effect of a small amount of sodium acetate added to the arginine formulation was tested. The purpose was to demonstrate the effectiveness of the addition of arginine to modify the insulin solubility at physiological pH in vivo, resulting in extended duration of action.

Study Design:

Insulin 0.45 IU/kg was administered by subcutaneous injection to diabetic induced miniature swine. On dose administration, pigs were fed 500 g of their normal diet, and blood insulin and glucose were monitored for the following 24 hours.

Insulin Test Formulations:

1. Insulin 100 U/ml+Zinc chloride 2.5 mg/ml+Arginine 2.5 mg/ml

2. Insulin 100 U/ml+Zinc chloride 2.5 mg/ml

3. Insulin glargine (Lantus)

4. Insulin 100 U/ml+Zinc chloride 2.5 mg/ml+Arginine 2.5 mg/ml+0.579 sodium acetate+0.5 mg/mL metacresol.

Results:

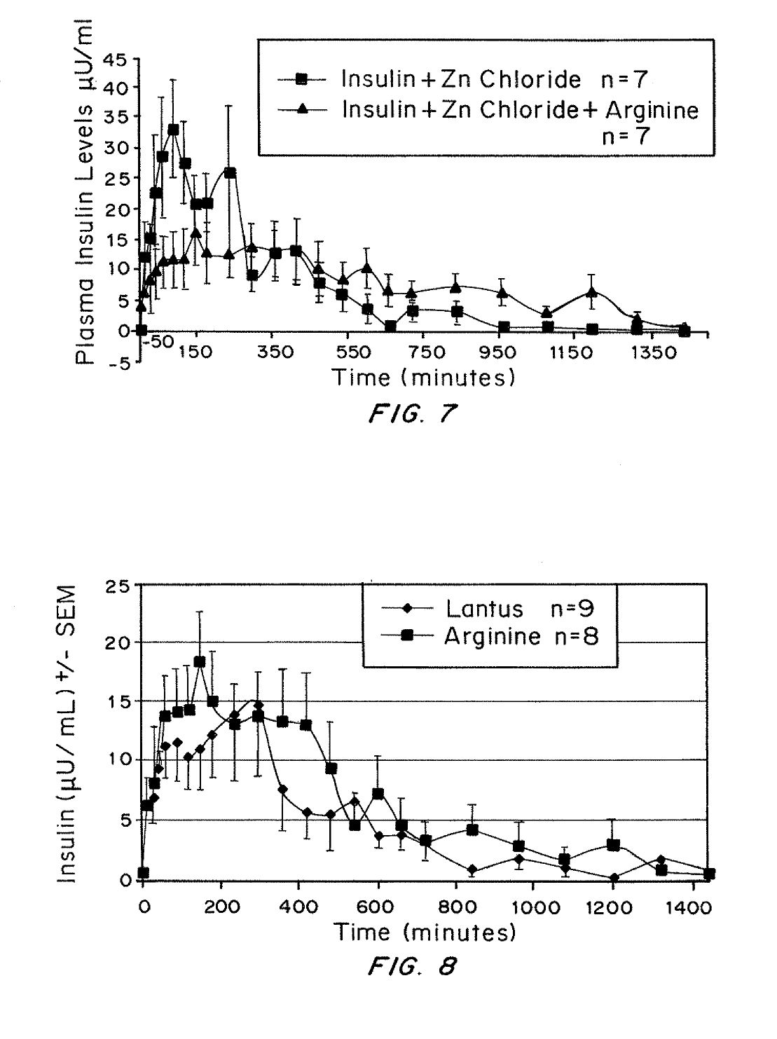

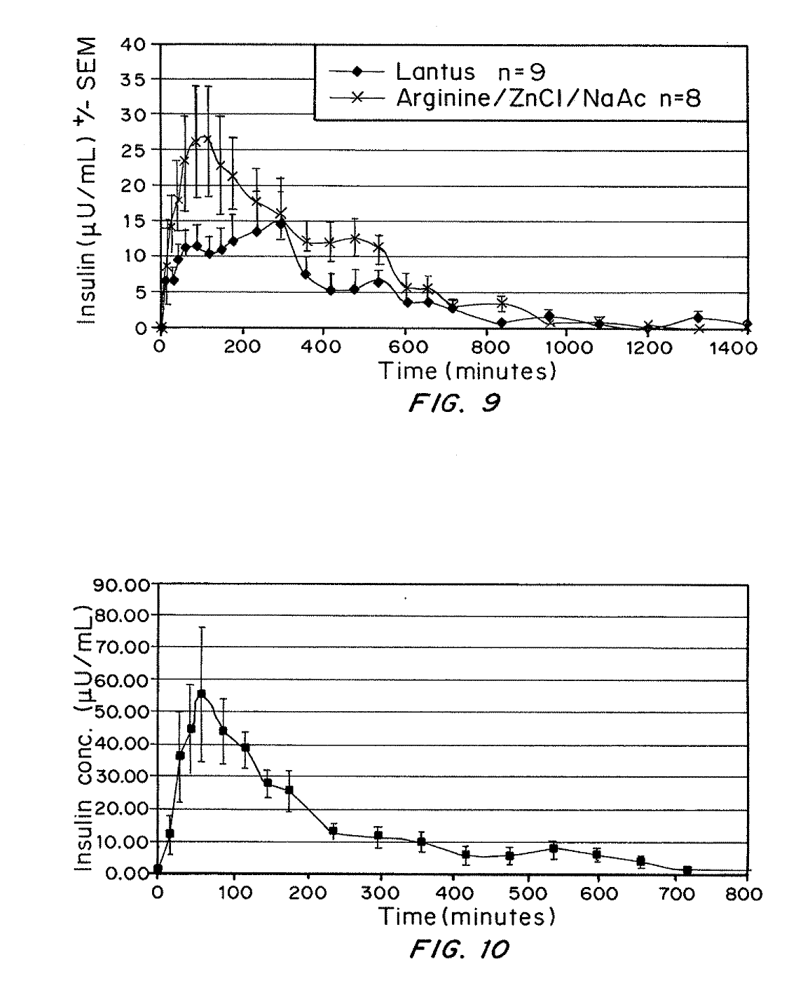

FIG. 7 is a graph of mean insulin concentration versus time of a subcutaneous injection of the test basal formulations #1 versus. #2 (see above). FIG. 8 is a graph of mean insulin concentration versus time of test basal formulations #1 versus #3 (see above) containing arginine as compared to Lantus. FIG. 9 is a graph of mean insulin concentration versus time of test basal formulation containing arginine and sodium acetate versus Lantus (#4 versus. 3#, see above).

### Example 7

### Prandial-Basal Profile in Miniature Diabetic Swine

Materials and Methods:

The purpose of this study was to determine if a combined insulin profile of a prandial (short acting) and basal (long acting) could be made in a single injectable formulation.

The insulin formulation contained regular human insulin 100 U/mL, histidine 0.5 mg/ml, and zinc acetate 2 mg/mL with salts added to adjust isotonicity and pH adjusted to 4. The sterile filtered formulation was subcutaneously injected in miniature diabetic swine at a dose of 0.45 U/kg. The animals were fed 500 g of food immediately after injection. Blood glucose and plasma insulin were monitored for the next 24 hours.

Results:

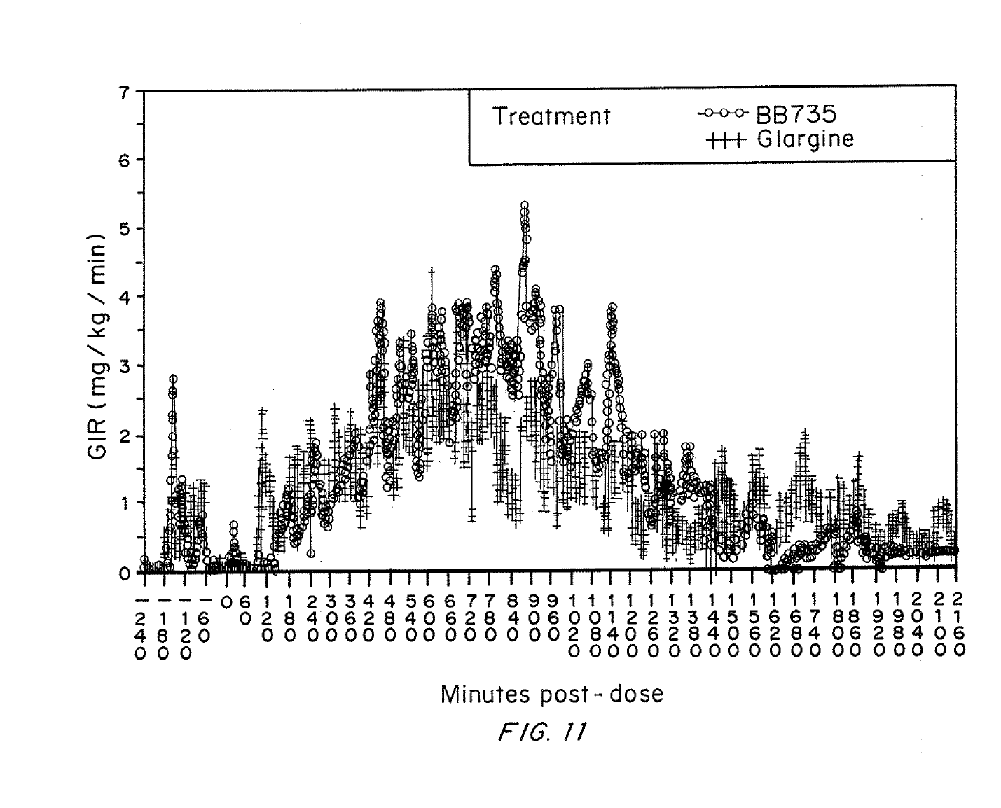

FIG. 10 shows mean the baseline subtracted plasma insulin concentration versus. time profile following the prandial/basal formulation injection containing histidine.

The histidine/zinc acetate insulin profile showed an initial burst early post injection, followed by a basal profile. Since the insulin level was sustained for up to 12 hours this formulation could be used for a prandial/basal application.

### Example 8

### Basal Formulation in Patients with Type 1 Diabetes

Materials and Methods

A single center, randomized, crossover, glucose clamp study was designed to evaluate the pharmacokinetic and pharmacodynamic properties of the new basal formulations in patients with type 1 diabetes. Three formulations were evaluated, one of which was composed of a sodium acetate formulation (100 IU insulin, 3 mg/mL ZnCl₂, 6 mg/mL NaAcetate).

Patients were randomly administered a dose of 0.5 U/kg of each study drug on each study day, including on one occasion insulin glargine (Lantus®). Each patient's glucose was first stabilized using the euglycemic clamp method and then the insulin dose was administered at time 0. Glucose was subsequently infused (GIR) to counteract insulin absorption as needed post injection throughout the 24 hour period.

Results

The mean glucose infusion rate (GIR) of six patients is shown in FIG. 11, comparing insulin glargine to the sodium acetate insulin formulation (735). The graph shows that the initial rate, peak GIR and duration of the sodium acetate insulin formulation is very similar to that of insulin glargine, indicating that the precipitation occurred post administration and had a subsequent slow release of insulin, to provide a near peakless basal profile, comparable to that insulin glargine.

Unless defined otherwise, all technical and scientific terms used herein have the same meanings as commonly understood by one of skill in the art to which the disclosed invention belongs. Publications cited herein and the materials for which they are cited are specifically incorporated by reference.

Modifications and variations of the methods and materials described herein will be obvious to those skilled in the art from the foregoing description and are intended to be encompassed by the following claims.

We claim:

1. A basal insulin formulation comprising a solution of recombinant human insulin at a pH between 3.5 and 4.5, preferably 3.8 to 4.2, or 7.5 to 8.5, optionally in combination with a stabilizing agent, buffering agent and precipitating agent, but not including protamine.

2. The formulation of claim 1, in the form of a clear solution having a pH greater than 7.5, which forms a precipitate at physiological pH.

3. The formulation of claim 1, further comprising an insulin analog.

4. The formulation of claim 1 comprising a stabilizing agent which maintains the insulin as a hexamer, preferably zinc at a concentration of 50 micrograms or less.

5. The formulation of claim 1 comprising precipitating agent selected from the group consisting of buffering agents, solubility modifying agents, precipitation seeding agents, and precipitation enhancing agents.

6. The formulation of claim 5, comprising a precipitation enhancing agent selected from the group consisting of zinc acetate, zinc oxide, zinc citrate, zinc carbonate, zinc sulfate, or zinc chloride, calcium chloride and other divalent salts used at non-toxic levels.

7. The formulation of claim 6 comprising zinc chloride in a concentration range of 0.1 to 10 mg/mL, most preferably 2-3 mg/mL.

8. The formulation of claim 5 wherein the precipitating agent is a buffering agent, preferably selected from the group consisting of acetate, citrate, phosphate, carbonate, and barbital, most preferably sodium acetate in a concentration in the range of 0.2 to 20mg/mL, preferably from 1 to 10 mg/mL, most preferably between 5 and 6 mg/mL.

9. The formulation of claim 5 wherein the precipitating agent is a solubility modifying agent, preferably a charged amino acid, more preferably selected from the group consisting of arginine, histidine, lysine, most preferably Arginine in the range of 0.005 to 10 mg/mL.

10. The formulation of claim 5 wherein the precipitating agent is a seeding agent selected from the group consisting of cysteine, L-proline and tyrosine, and nanoparticles such as C₆₀ or Au₁₂.

11. The formulation of claim 1, comprising at least one pH modifying agent selected from the group consisting of sodium hydroxide, citric acid, hydrochloric acid, acetic acid, phosphoric acid, succinic acid, sodium hydroxide, potassium hydroxide, ammonium hydroxide, magnesium oxide, calcium hydroxide, calcium carbonate, magnesium carbonate, magnesium aluminum silicates, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof

12. The formulation of claim 1, containing a preservative.

13. The formulation of claim 1, provided in a kit consisting of two or more containers which are mixed at the time of administration to form an insulin solution at the time of injection.

14. The formulation of claim 1, providing a basal effective amount of insulin for a period of 12 to 24 hours following administered by subcutaneous, intramuscular, or intradermal injection.

15. The formulation of claim 1 providing an initial burst release of insulin.

16. The formulation of claim 15 providing sustained release of insulin over a period of 12 to 24 hours after an initial burst release.

17. The formulation of claim 1 providing a insulin basal release profile for a short, medium or long duration, preferably of 12 to 16 hours, 16 to 20, or 20 to 24 hours.

18. A method of providing a basal insulin to an individual in need thereof comprising administering the formulation of claim 1.

19. The method of claim 18 wherein the insulin is provided in a first container as a lyophilized powder which is reconstituted at the time of administration and the other ingredients are present in one or both of the vials.

20. The method of claim 19 wherein the contents of the two containers are mixed to form a clear solution prior to administration.
